(19) United States
(12) Patent Application Publication
(10) Pub. No.: US 2016/0360984 A1
(43) Pub. Date: Dec. 15, 2016

(54) Optical Photoplethysmogram Signal Shape Feature Biological Monitor

(71) Applicant: Microsoft Technology Licensing, LLC, Redmond, WA (US)

(72) Inventors: Haithem Albadawi, Redmond, WA (US); Farah Shariff, Kirkland, WA (US); Zongyi Liu, Redmond, WA (US)

(21) Appl. No.: 14/740,067

(22) Filed: Jun. 15, 2015

(51) Int. Cl. A61B 5/024; A61B 5/00

(52) U.S. Cl. CPC A61B 5/02433; A61B 5/742; A61B 5/7246; A61B 5/7221; A61B 2562/0238

(57) ABSTRACT

A system is described for providing the value of a biological characteristic. The system includes a sensor. The sensor is configured to generate a time domain photoplethysmogram (PPG) input signal. The system further includes a first computation module coupled to the sensor. The first computation module is configured to evaluate portions of the generated time domain PPG signal against predetermined quality criteria. The system further includes a second computation module coupled to the first computation module. The second computation module is configured to use portions of the generated time domain PPG signal that meet the predetermined quality criteria to generate an output value. The output value characterizes a biological characteristic at a point in time.

### BACKGROUND

### Background and Relevant Art

Most individuals have some interest in monitoring and improving their health. Previously such individuals have been limited to monitoring health based on perceived energy levels, sickness symptoms, weight, and general appearance. However, in recent times, various electronic monitoring devices have assisted the individual in monitoring and quantifying factors that were previously not able to be easily monitored and quantified. One such example is the ability to measure heart rate.

Individuals have been able to purchase home use heart rate monitors for some time. For example, individuals have been able to obtain a system that includes a chest strap that the individual attaches around the chest with sensors that contact the skin on the chest near the heart. The sensors detect electrical signals produced by the body that cause the heart to beat and transmit information about the electrical signals to a monitor. Such monitors are often embodied in wrist watches. While these types of systems are highly accurate, they are also quite bulky and may not be comfortable or fashionable for all-day, everyday use.

However, more recently, devices have been available that use photoplethysmograms (PPGs), which are optically obtained representations of volumetric measurements of an organ. Such systems function by illuminating a surface, such as the skin near a blood vessel or artery, and measuring changes in light absorption. For example, light absorption may change due to changes in blood pressure (such as due to a heartbeat), changes in blood oxygen levels, and changes in blood glucose levels. Such systems can eliminate the need for a chest strap, as the illumination source (typically including an LED) and the detector (typically including a photodiode) can be incorporated directly in the monitor, such as a wristwatch. However, such systems are susceptible to errors.

However, errors may be introduced by the wearer of such a system. For example, an individual moving can change the amount of light absorption, limb positioning can change the amount of light absorption, muscle tightening can change the amount of light absorption, movement of the device on the skin can change the amount of light absorption, improper wearing of the device can change the amount of light absorption, even something as simple as pressing on the skin, which causes a temporary lightening of the skin, can cause a change in the amount of light absorption.

Additionally, hardware issues may change the amount of light absorption detected. For example, such devices may have other functionality such as selectable illumination of a display and vibrating alert mechanisms. When one of these other functionalities is activated, power may be reduced to the sensors monitoring the change in light absorption as a result of other hardware consuming power. This may cause a change in the amount of light absorption detected.

Today, most algorithms evaluate the quality of PPG signals using an FFT (Fast Fourier Transform)/auto-correlation based spectrogram, which is a frequency domain based analysis of a PPG signal. However, such an approach requires approximately five to ten seconds of data buffering and one or more bad samples taken during the data buffering may pollute an entire buffer filled with data. Thus, such systems are not able to perform a fast lock of a biological characteristic (such as heart rate), and even when they do lock, such systems may have significant errors in biological characteristic reporting.

The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

One embodiment illustrated herein includes a method for performing adaptive filtering of a time domain photoplethysmogram (PPG) input signal. The method includes generating a time domain PPG signal using a sensor. The method further includes evaluating portions of the generated time domain PPG signal against predetermined quality criteria. The method also includes using portions of the generated time domain PPG signal that meet the predetermined quality criteria to generate an output value. The output value characterizes a biological characteristic at a point in time.

Another embodiment includes a system for providing a value of a biological characteristic. The system includes a sensor. The sensor is configured to generate a time domain photoplethysmogram (PPG) input signal. The system further includes a first computation module coupled to the sensor. The first computation module is configured to evaluate portions of the generated time domain PPG signal against predetermined quality criteria. The system further includes a second computation module coupled to the first computation module. The second computation module is configured to use portions of the generated time domain PPG signal that meet the predetermined quality criteria to generate an output value. The output value characterizes a biological characteristic at a point in time.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be obvious from the description, or may be learned by the practice of the teachings herein. Features and advantages of the invention may be realized and obtained by means of the instruments and combinations particularly pointed out in the appended claims. Features of the present invention will become more fully apparent from the following description and appended claims, or may be learned by the practice of the invention as set forth hereinafter.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not therefore to be considered to be limiting in scope, embodiments will be described and explained with additional specificity and detail through the use of the accompanying drawings in which:

FIG. 1 illustrates a system for evaluating shape features of a time domain photoplethysmogram signal;

FIG. 2 illustrates another system for evaluating shape features of a time domain photoplethysmogram signal;

FIG. 3 illustrates another system for evaluating shape features of a time domain photoplethysmogram signal; and

FIG. 4 illustrates a method for performing adaptive filtering of a time domain photoplethysmogram input signal.

### DETAILED DESCRIPTION

Some embodiments described herein implement a system that uses the shape features of a photoplethysmogram (PPG) signal to estimate the signal quality of the PPG signal. Biological characteristic (such as heart rate) reporting can be improved based on the estimated signal quality. In the following examples, heart rate is used, but it should be appreciated that the principles can be applied to other biological characteristics such as blood oxygen levels, glucose levels, blood pressure, etc.

One way that biological characteristic reporting improvement can be done is by discarding low-quality shape features of a PPG signal. More generally, a shape feature is a portion of a time domain PPG signal. In some embodiments each shape feature is defined by zero-crossings of the PPG signal. In particular, a shape feature of a PPG signal may be the portions of the PPG signal that occur between the upward crossings of a zero axis on which the PPG signal is graphed. Thus, each shape feature in this example represents a cycle. A shape feature will typically correspond to a biological cycle, such as a complete heartbeat cycle. The system buffers only representations of shape features of the PPG signal that meet or exceed one or more predetermined quality measures while discarding shape features of the PPG signal that do not meet the predetermined quality measures. The buffered shape features are then used to generate a biological characteristic value. Thus, for example, for a new detected zero-crossing of a PPG signal, embodiments compute the quality of a shape feature of the PPG signal associated with the new detected zero-crossing. If the quality is “low” (defined as a shape feature that does not meet predetermined quality criteria), then embodiments discard the shape feature so that it will not pollute a buffer. If the quality is “high” (defined as a shape feature that meets predetermined quality criteria), then embodiments add a representation of the shape feature of the PPG signal to the buffer where it is used to generate a digital characterization of a biological characteristic, such as a biological characteristic value. For example, such a value may be a heart rate.

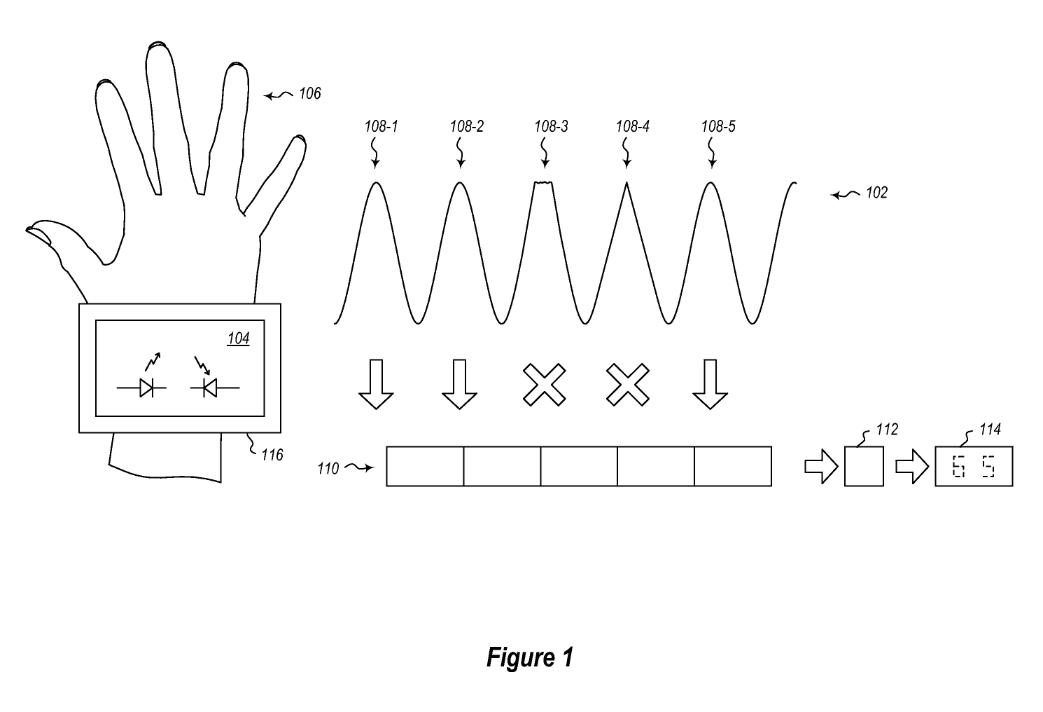

FIG. 1 illustrates a PPG signal 102 generated using a sensor 104 placed on a user's skin 106. The PPG signal 102 is shown with five shape features 108-1, 108-2, 108-3, 108-4 and 108-5, where each shape feature is defined by zero crossings, as will be illustrated in more detail below. As each shape feature is generated by the sensor 104, it is evaluated to determine whether or not it meets certain quality criteria. Examples of various criteria are illustrated in more detail below. If a shape feature meets the quality criteria, a representation of that shape feature is added to a buffer 110. The contents of the buffer 110 are used by a biological characteristic computation module 112 to generate a biological characteristic value. In the example illustrated, the biological characteristic value is displayed on a display 114. In some embodiments, the display 114 may be part of a wearable device 116, such as a wristwatch, that includes both the sensor 104 and the display 114.

In the example illustrated in FIG. 1, representation of shape features 108-1, 108-2, and 108-4 are added to the buffer 110 while shape features 108-3 and 108-4 are discarded for not meeting the quality criteria. In particular, shape feature gradient that exceeds an allowed range of gradients (i.e. it has a slope gradient that rises too quickly to the peak to be considered of good quality). These are two examples of quality criteria, but as will be illustrated in more detail below, other criteria may alternatively or additionally be used.

Another way that biological characteristic reporting improvement can be achieved is by detecting a series of consecutive shape features of a PPG signal that are all “high quality” (defined as meeting predetermined quality criteria). When such a series is detected, the entire buffer can be flushed and replaced with representations of shape features of the PPG signal in the series, which can be used to create a highly accurate characterization of a biological characteristic. Thus for example, if embodiments detect N shape features of a PPG signal with high qualities consecutively (where, for example, N=5 in some embodiments), then embodiments perform a fast sync by purging the values of the buffer and refilling it with the mean value of the N shape features. In some embodiments, for this functionality, enhanced predetermined quality criteria are used to ensure that very high quality shape features are used.

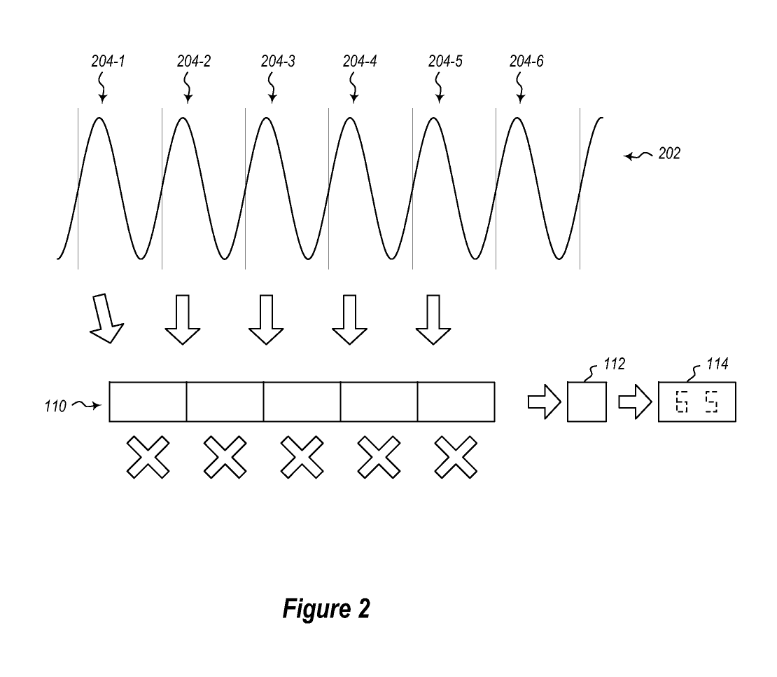

FIG. 2 illustrates a PPG signal 202 that may be obtained in a fashion similar to that illustrated in FIG. 1 by using a sensor 104 placed on the skin 106 of a user. In this example, embodiments determine that all five shape features 204-1, 204-2, 204-3, 204-4 and 204-5 meet predetermined quality criteria and that the five shape features are consecutive in the PPG signal 202, with no shape features of the PPG signal 202 not meeting the predetermined quality criteria intervening between any of the five shape features 204-1, 204-2, 204-3, 204-4 and 204-5. When this condition is detected, the buffer 110 is purged (as represented by the five X's below the buffer 110). This removes previous representations of shape features of the PPG signal 202. Representations of each of the five shape features 204-1, 204-2, 204-3, 204-4 and 204-5 are then added to the buffer 110. The representations from the buffer 110 are used by the biological characteristic computation module 112 to compute a biological characteristic value. This value can be output in various fashions. In the example illustrated, the biological characteristic value is output to the display 114. However, the value could be provided additionally or alternatively to a storage system for archival or to other systems that can make use of the value.

Illustrating now additional details that can be used in the various embodiments, as illustrated above, embodiments use time-domain based shape features to evaluate the PPG signal quality, which is different from the traditional approaches that use the frequency-domain based signal to noise ratios (SNRs). As such, embodiments can be implemented in a fashion where signal quality is computed based on the shape feature of each single zero crossing, in contrast to a frequency domain computation which might require at least five seconds of data (equating to 5 zero crossings for a heart rate of 60 bpm). Embodiments can be configured to determine the signal quality by comparing the shape features of multiple zero-crossings to see if the features are clustered by similarity measures to each other. Compared with frequency domain features, the shape features are much cheaper to operate on in terms of both computation and memory, and thus power consumption is reduced when using the shape features. For example, in some embodiments, a PPG sensor sampling rate might be 60 Hz. Five seconds of PPG data will need 600 bytes (2 bytes per sample). In contrast, five zero-crossing shape features only costs 55 bytes (I bytes per zero-crossing).

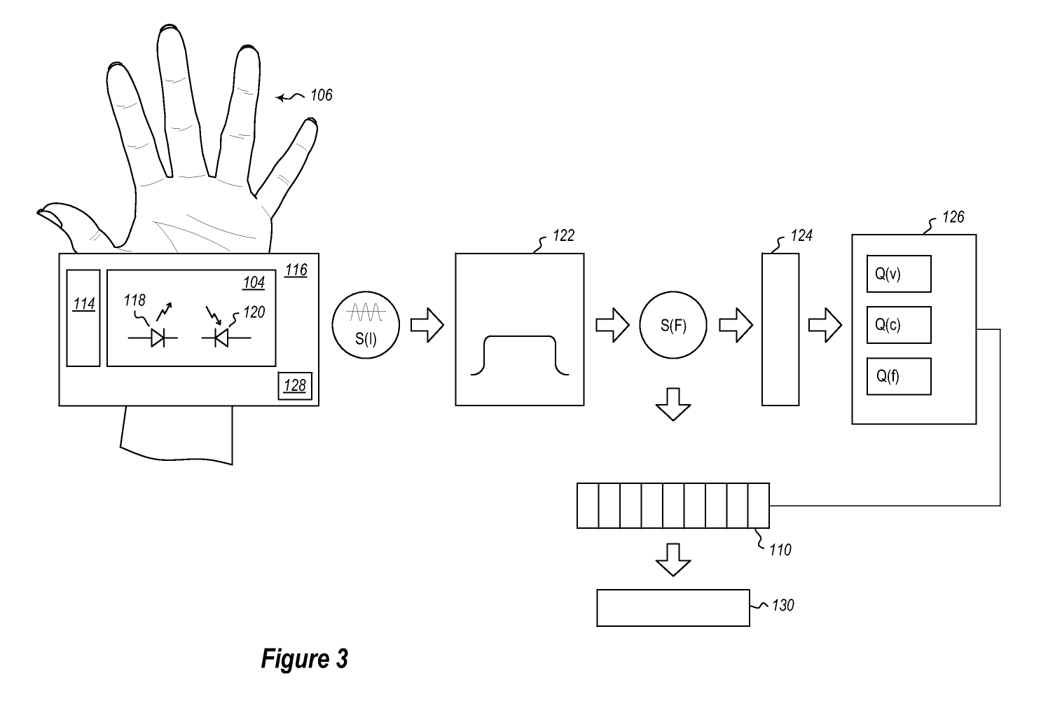

FIG. 3 illustrates a wearable device 116. The wearable device 116 includes a sensor 104. The sensor 104 is coupled to a user's skin 106, such as by wearing a wristwatch with the sensor 104 included in it. In the illustrated example, the sensor includes an LED 118 and a photodiode 120. The LED 118 can illuminate the user's skin 106 and the photodiode 120 can be used to determine the amount of absorption of light from the LED 118 that occurs. This can be used to generate a PPG signal, such as signals 102 and 202 illustrated in FIGS. 1 and 2. As illustrated in the examples herein, the PPG signal is represented as S(I).

FIG. 3 illustrates that a shape feature filter 122 takes S(I) as input and pre-processes the signal S(I) using a fixed band-pass filter that outputs the time domain PPG signal S(F). The band pass filter is based on reasonable parameters for the biological characteristic being measured. Thus, for example, if the wearable device 116 is attempting to monitor heart rate, the band pass filter may filter between frequencies of 30 to 180 Hz or 30 to 220 Hz as these represent reasonable, but comprehensive, frequency ranges for a heartbeat.

When a new zero-crossing T(ZC) is detected for the time domain PPG signal S(F), a shape feature computation module 124 computes a number characteristics of shape features as illustrated below. In the examples illustrated, this is performed using the time domain vector I, where I=[S(F)(t−T(ZC)), S(F)(t−T(ZC)+1), . . . , S(F)(t)], and its height normalized vector I′ where max(I′)−min(I′)=200. Note that the FIG. 200 used in this equation is based on an empirical computation and distribution comparison using samples of a particular set of hardware, particularly, the hardware in the Band available from Microsoft, Corporation of Redmond, Wash. Other hardware may have a different value. Note that in the embodiments used herein a zero-crossing will define a shape feature of a time domain PPG signal being evaluated. The shape features are defined as one cycle, and thus are based on one upward zero crossing to the next upward zero crossing. In the examples shown in FIGS. 1 and 2, the PPG signals 102 and 202 are divided into shape features from one upward zero crossing to the next upward zero crossing. Note that in other embodiments, other measures may be used to define shape features of the PPG signal. The following shape features for shape features of a PPG signal are computed:

Height: The height of a shape feature is computed as max(I)−min(l).

Number of turning points: A turning point occurs when signal changes from moving positive to moving negative or vice versa.

Number of peaks: The number of peaks is defined as the number of local maxima points in a shape feature where slope is equal to 0. This can be determined by taking a first order derivative of a time domain PPG signal.

Number of samples: The number of samples for a shape feature of a PPG signal is computed as the length of the vector I.

Up slope count (UpSlopeCount): The up slope count is computed as the number of samples going in an upward direction (e.g., having a positive slope) as determined by a first order derivative where I′>T. This can be conceptualized as the time domain PPG signal S(F) increasing over time.

Down slope count (DownSlopeCount): The down slope count is the number of samples going in a downward direction (e.g., having a negative slope) as determined by a first order derivative where I′<−T. This can be conceptualized as the time domain PPG signal S(F) decreasing over time.

Flat count (FlatCount): The flat count is the number of samples that are flat as determined by a first order derivative where I′ is between [−T, T]. This can be conceptualized as points on the time domain PPG signal that are neither increasing nor decreasing (e.g., where the slope is zero).

Max Absolute Gradient (maxAbsGradient): The max absolute gradient is the maximum value of the first order derivative of I′ (in other words, the second order derivative of 1). This can be conceptualized as an indication of how quickly a time domain PPG signal S(F) is changing, either increasing or decreasing. Using this measure, shape features of time domain PPG signals that increase or decrease too quickly or too slowly to be valid measurements of a biological characteristic of interest can be identified.

In the particular embodiment illustrated in this example, using one or more characteristics of shape features identified by the shape feature computation module 124, a quality computation module 126 computes three quality values referred to herein as shape feature value quality, shape feature change quality, and shape feature fast sync quality. These are described in more detail below. It should be noted that embodiments can compute various different characteristics of shape features and quality measures and that embodiments of the invention are not limited to just the examples illustrated here. Other quality measures and comparisons can be used is other embodiments of the invention.

Shape feature value quality (Q(V)) is computed using the characteristics of the current shape feature. Specifically, Q(V) for a shape feature of a time domain PPG signal is “low” (i.e., does not meet quality criteria) if the sensor 104 is not experiencing significant movement as indicated by an accelerometer 128, and one of the three conditions below is true:

(1). Max Absolute Gradient is outside of a predetermined range. For example, using the hardware available in the Microsoft Band available from Microsoft Corporation of Redmond, Wash., the range may be from 10 to 60. Thus, in one particular embodiment using such hardware, the particular shape feature of the PPG signal does not meet shape feature value quality criteria when the max absolute gradient is <10 or >60

(2). Number of turning points is outside of a predetermined range. For example, using the hardware available in the Microsoft Band available from Microsoft Corporation of Redmond, Wash., the range may be from 1 to 3. Thus, in one particular embodiment using such hardware, the particular shape feature of the PPG signal does not meet shape feature value quality criteria when the number of turning points in a shape feature of a PPG signal is <1 or >3.

(3). Number of peaks is different than an allowed number of peaks. For example, using the hardware available in the Microsoft Band available from Microsoft Corporation of Redmond, Wash., a single peak is needed and only a single peak is allowed. Thus, when the number of peaks is !=1, then the particular shape feature of the PPG signal does not meet shape feature value quality criteria.

Shape feature change quality (Q(C)) is computed by comparing the characteristics of a shape feature of the time domain PPG signal being evaluated (e.g., the current zero-crossing (ZC(Now))) with other shape features of the time domain PPG signal (e.g., the most recent five zero-crossings (ZC(Hist))). For example, with reference to FIG. 2, characteristics of the shape feature 204-6 may be computed. Those characteristics can then be compared with the same computed characteristics of shape features 204-1, 204-2, 204-3, 204-4, and 204-5. If the characteristics of shape feature 204-6 are similar enough to the combined characteristics of shape features 204-1, 204-2, 204-3, 204-4, and 204-5 as defined by some predetermined quality criteria, then the Q(C) for the shape feature 204-6 is considered “high.” The following illustrates a set of computations and evaluation against criteria that may be performed:

(1). Compute the standard deviation (std) and mean of ZC(Hist) for each shape feature. For example, embodiments may compute the height for a shape feature of a PPG signal as given by std(Height(Hist)).

(2). Compute the normalized standard deviation (nstd) of ZC(Hist) for each shape feature, where nstd(i)=std(i)/mean(i).

(3). Compute the distance of ZC(Now) and the mean of ZC(Hist) for each shape feature. For example, the distance may be given by dist(height)=height(Now)−mean(height(Hist))

(4). Report quality low if any of the conditions below is true (note that the actual values used here are for example purposes of embodiments implemented using the hardware available in the Microsoft Band available from Microsoft Corporation of Redmond, Wash. but that other systems may use other values, or even different measures as appropriate):

(a). nstd(height(Hist))<0.1 and dist(height)/mean(Height(Hist))>1.0

(b). std(maxAbsGradient(hist))<1.0 and dist(maxAbsGradient)>8.0

(c) std(Number of samples (hist))<1.0 and dist(number of samples)>8.0

(d) max(std(UpSlopeCount), std(DownSlopeCount), std(FlatCount))<2.0 and max(dist(UpSlopeCount), dist(DownSlopeCount), dist(FlatCount))>8.0

(5). If none of the conditions in (a)-(d) is true (all the std and nstd listed are below the specified thresholds), then embodiments report quality high, otherwise embodiments report quality medium. Medium quality samples can be added to the buffer 110, but will not be used for a fast sync.

Shape feature fast sync quality (Q(F)) is similar to Q(C) but with stricter criteria. Specifically, it computes the stats for the most recent five zero-crossings ZC(Hist):

(1). Compute the standard deviation (std) and mean of ZC(Hist) for each shape feature (e.g., each shape feature of the PPG signal).

(2). Compute the maximum distance (D) and the normalized distance (ND) of ZC(Hist) for each shape feature, where ND(i)=D(i)/mean(i)

(3). Report fast sync quality as true if ALL of the conditions below are true (for the example illustrated, noting that in other embodiments using other hardware, other criteria may be specified):

(a). max(ND(UpSlopeCount), ND(DownSlopeCount), ND(FlatCount))<=1.0 and max(std(UpSlopeCount), std(DownSlopeCount), std(FlatCount))<=4.0.

(b). ND(Number of samples)<=0.4 and D(Number of samples)<=10.

(c). ND(height)<=0.5.

Thus, embodiments may be implemented as follows:

When a new zero-crossing is detected (indicating a new shape feature or shape feature of the PPG signal to be evaluated) by the shape feature computation module 124, the shape feature computation module 124 and the quality computation module 126 are used to compute the Q(V) and Q(C) for the shape feature of the PPG signal. If either of them is “low” by not meeting predetermined quality criteria, such as those illustrated above, then embodiments discard that shape feature of the PPG signal by not buffering a representation of the shape feature so that low quality samples will not pollute the buffer 110, which contains values to compute a biological characteristic. Any shape features of the PPG signal that meet the criteria, and thus result in Q(V) and Q(C) that are “high” quality, can have a representation placed in the buffer 110 which can be used by a biological characteristic computation module 130 to compute a value for a biological characteristic. The computed values for the biological characteristic can then be stored, used for further analysis of health, displayed directly to a user on a display 114, otherwise used, or combinations thereof.

At any time when a Q(F) is found that is high, a fast sync may be performed by purging the buffer 110 completely, and re-filling it with the mean value of ZC(Hist). As in the illustrated example, Q(F) only needs 5 samples, which is 5 seconds when the HR is 60 bpm. Using these samples, a system can recover quickly from an event that may cause computed biological characteristic values to be inaccurate or unable to be calculated due to lack of high quality shape features of the PPG signal.

The following discussion now refers to a number of methods and method acts that may be performed. Although the method acts may be discussed in a certain order or illustrated in a flow chart as occurring in a particular order, no particular ordering is required unless specifically stated, or required because an act is dependent on another act being completed prior to the act being performed.

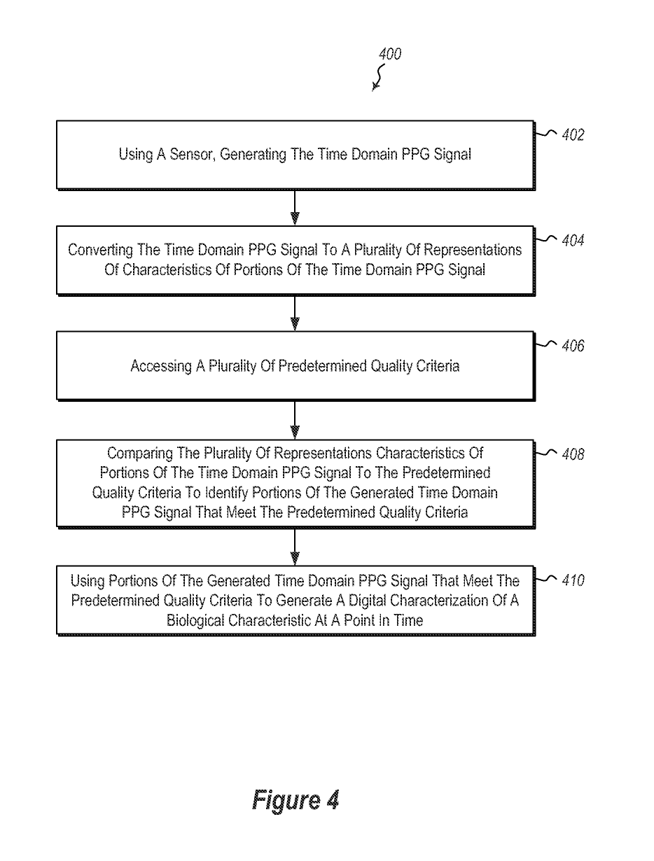

Referring now to FIG. 4, a method 400 is illustrated. The method includes acts for performing adaptive filtering of a time domain photoplethysmogram (PPG) signal. The method includes generating a time domain PPG signal (act 402) using a sensor. For example, the sensor 104 illustrated in FIG. 1 may be used to generate the PPG signal 102. In some embodiments, the sensor 104 may include an LED 118 and a photodiode 120. In some embodiments, the LED 118 may be a green LED, an infrared LED, or other appropriate LED.

The method 400 further includes converting the time domain PPG signal to a plurality of representations of characteristics of portions of the time domain PPG signal (act 404). For example, the time domain signal may be converted to gradient representations of the time domain PPG signal, turning point representations; peak representations, historical statistical representations; etc.

The method 400 further includes accessing a plurality of predetermined quality criteria (act 406). For example, the predetermined quality criteria may be related to gradient ranges, turning point ranges, peaks, historical statistical criteria, etc.

The method 400 further includes comparing the plurality of representations characteristics of portions of the time domain PPG signal to the predetermined quality criteria to identify portions of the generated time domain PPG signal that meet the predetermined quality criteria (act 408). For example, FIG. 3 illustrates that a quality computation module 126 can be used to compute characteristics of portions (where an example of a portion is a shape feature as illustrated above) of PPG signals and to compare those characteristics to certain predetermined quality criteria.

The method 400 further includes using portions of the generated time domain PPG signal that meet the predetermined quality criteria to generate a digital characterization of a biological characteristic at a point in time (act 410). As illustrated in FIG. 3, representations of portions of the PPG signal can be stored in the buffer 110 and be used by a biological characteristic computation module 130 to compute a biological characteristic value. Such biological characteristics may include, for example, heart rate, blood oxygen levels, and glucose levels.

The method 400 may be practiced where the predetermined quality criteria comprise a gradient range. In this example, converting the time domain PPG signal to a plurality of representations of characteristics of portions of the time domain PPG signal comprises converting the time domain PPG signal to a gradient representation of the time domain PPG signal by computing a second order derivative of the time domain PPG signal. In this example, comparing the plurality of representations of characteristics of portions of the time domain PPG signal to the predetermined quality criteria comprises comparing the second order derivative of the generated time domain PPG signal to the gradient range.

The method 400 may be practiced where the predetermined quality criteria includes a range of turning points. In such embodiments, comparing the plurality of representations characteristics of portions of the time domain PPG signal to the predetermined quality criteria comprises, for each portion, comparing a number of turning points for the portion to the range of turning points in the predetermined quality criteria. Turning points can be calculated by using a first order derivative of the generated time domain PPG signal. In particular when the derivative changes from positive to negative or vice versa, a turning point is detected.

The method 400 may be practiced where the predetermined quality criteria includes a number of peaks. In such embodiments, comparing the plurality of representations characteristics of portions of the time domain PPG signal to the predetermined quality criteria includes, for each portion, comparing a number of peaks in the portion to the number of peaks in the predetermined quality criteria. The number of peaks can be determined by using a first order derivative of the generated time domain PPG signal. In particular, when the first order derivative is 0, a peak is detected.

The method 400 may be practiced where the predetermined quality criteria comprises one or more predetermined statistical similarity measure limits between portions of the generated time domain PPG signal. In such embodiments, comparing the plurality of representations characteristics of portions of the time domain PPG signal to the predetermined quality criteria includes, for each portion, computing a statistical similarity between the portion of the generated time domain PPG signal and previous portions of the generated time domain PPG signal and determining if the computed statistical similarity measure is within the predetermined statistical similarity measure limit. Thus, for example as illustrated in FIG. 3, the shape feature 204-6 may be compared (individually, selectively, or as an aggregation) to previous shape features 204-1 through 204-5.

The method 400 may further include adding representations of portions of the generated time domain PPG signal that meet the predetermined quality criteria to a buffer. Portions of the generated time domain PPG signal that do not meet the predetermined quality criteria are discarded so as to avoid having low quality PPG signal representations in the buffer. Representations of portions of the generated time domain PPG signal in the buffer are used to generate the digital characterization of the biological characteristic at the point in time. Examples of this are illustrated in FIGS. 1, 2 and 3 which make use of the buffer 110.

Embodiments of the method 400 practiced using a buffer may further include identifying a plurality of consecutive portions of the time domain PPG signal that meet additional predetermined quality criteria. Based on identifying a plurality of consecutive portions that meet additional predetermined quality criteria, the buffer is purged and filled with representations of the plurality of consecutive portions. Generating a digital characterization of the biological characteristic is performed by using the representations of the plurality of consecutive portions in the buffer. An example of this is illustrated in FIG. 2, where the buffer 110 is purged and filled with representations of the shape features 204-1 through 204-5. Note that individual representations of individual shape features can be added to the buffer 110, but in some embodiments, it may be more efficient to add an aggregated representation of a plurality of individual shape feature to a plurality of different buffer locations in the buffer 110. Thus, for example, a single aggregated representation (e.g. an average or mean) of the shape features 204-1 through 204-5 could be added to five (in the current example) different buffer locations of the buffer 110. In some such embodiments, the additional predetermined quality criteria may be a set of criteria with stricter parameters than the predetermined quality criteria. This can be used to perform a fast lock onto a biological characteristic value.

Further, the methods may be practiced by a computer system including one or more processors and computer-readable media such as computer memory. In particular, the computer memory may store computer-executable instructions that when executed by one or more processors cause various functions to be performed, such as the acts recited in the embodiments.

Embodiments of the present invention may comprise or utilize a special purpose or general-purpose computer including computer hardware, as discussed in greater detail below. Embodiments within the scope of the present invention also include physical and other computer-readable media for carrying or storing computer-executable instructions and/or data structures. Such computer-readable media can be any available media that can be accessed by a general purpose or special purpose computer system. Computer-readable media that store computer-executable instructions are physical storage media. Computer-readable media that carry computer-executable instructions are transmission media. Thus, by way of example, and not limitation, embodiments of the invention can comprise at least two distinctly different kinds of computer-readable media: physical computer-readable storage media and transmission computer-readable media.

Physical computer-readable storage media includes RAM, ROM, EEPROM, CD-ROM or other optical disk storage (such as CDs, DVDs, etc), magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry or desired program code means in the form of computer-executable instructions or data structures and which can be accessed by a general purpose or special purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RAM within a network interface module (e.g., a “NIC”), and then eventually transferred to computer system RAM and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general purpose computer, special purpose computer, or special purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, and the like. The invention may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Alternatively, or in addition, the functionally described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), etc.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A system for providing a value of a biological characteristic, the system comprising:
a sensor, wherein the sensor is configured to generate a time domain photoplethysmogram (PPG) input signal;
a first computation module coupled to the sensor, wherein the first computation module is configured to evaluate portions of the generated time domain PPG signal against predetermined quality criteria; and
a second computation module coupled to the first computation module, wherein the second computation module is configured to use portions of the generated time domain PPG signal that meet the predetermined quality criteria to generate an output value, the output value characterizing a biological characteristic at a point in time.

2. The system of claim 1, wherein the first computation module is configured to compute gradients for the portions of the generated time domain PPG signal and to compare the computed gradients to a predetermined gradient range to evaluate the portions of the PPG signal against the predetermined quality criteria.

3. The system of claim 1, wherein the first computation module is configured to compute a number of turning points for each of the portions of the generated time domain PPG signal and to compare the number of computed turning points to a predetermined range of turning points to evaluate the portions of the PPG signal against the predetermined quality criteria.

4. The system of claim 1, wherein the first computation module is configured to compute a number of peaks for each of the portions of the generated time domain PPG signal and to compare the number of computed peaks for each portion of the time domain PPG signal to a predetermined number of peaks to evaluate the portions of the PPG signal against the predetermined quality criteria.

5. The system of claim 1, wherein the first computation module is configured to compute one or more statistical similarity measures between portions of the generated time domain PPG signal and to compare the computed one or more statistical similarity measures to one or more statistical similarity measure limits peaks to evaluate the portions of the PPG signal against the predetermined quality criteria.

6. The system of claim 1, further comprising a buffer coupled to the first computation module and the second computation module, wherein the buffer is configured to store representations of portions of the generated time domain PPG signal that meet the predetermined quality criteria, and wherein representations of portions of the generated time domain PPG signal in the buffer are used by the second computation module to generate the output value characterizing the biological characteristic at the point in time.

7. The system of claim 6, wherein the first computation module is configured to:
identify a plurality of consecutive portions that meet additional predetermined quality criteria;
based on identifying a plurality of consecutive portions that meet additional predetermined quality criteria:
cause the buffer to be purged; and
cause the buffer to be filled with representations of the plurality of consecutive portions.

8. The system of claim 1, wherein the sensor comprises an LED and a photodiode.

9. The system of claim 8, wherein the LED is a green LED.

10. The system of claim 8, wherein the LED is an infrared LED.

11. The system of claim 1, further comprising a display coupled to the second computation module, wherein the display is configured to display a value characterizing the biological characteristic.

12. A method of performing adaptive filtering of a time domain photoplethysmogram (PPG) signal, the method comprising:
using a sensor, generating the time domain PPG signal;
converting the time domain PPG signal to a plurality of representations of characteristics of portions of the time domain PPG signal;
accessing a plurality of predetermined quality criteria;
comparing the plurality of representations characteristics of portions of the time domain PPG signal to the predetermined quality criteria to identify portions of the generated time domain PPG signal that meet the predetermined quality criteria; and
using portions of the generated time domain PPG signal that meet the predetermined quality criteria to generate a digital characterization of a biological characteristic at a point in time.

13. The method of claim 12, wherein the predetermined quality criteria comprise a gradient range, and wherein converting the time domain PPG signal to a plurality of representations of characteristics of portions of the time domain PPG signal comprises converting the time domain PPG signal to a gradient representation of the time domain PPG signal by computing a second order derivative of the time domain PPG signal, and wherein comparing the plurality of representations of characteristics of portions of the time domain PPG signal to the predetermined quality criteria comprises comparing the second order derivative of the generated time domain PPG signal to the gradient range.

14. The method of claim 12, wherein the predetermined quality criteria comprise a range of turning points, and wherein comparing the plurality of representations characteristics of portions of the time domain PPG signal to the predetermined quality criteria comprises, for each portion, comparing a number of turning points for the portion to the range of turning points.

15. The method of claim 12, wherein the predetermined quality criteria comprise a number of peaks, and wherein comparing the plurality of representations characteristics of portions of the time domain PPG signal to the predetermined quality criteria comprises, for each portion, comparing a number of peaks in the portion to the number of peaks in the predetermined quality criteria.

16. The method of claim 12, wherein the predetermined quality criteria comprises one or more predetermined statistical similarity measure limits between portions of the generated time domain PPG signal and wherein comparing the plurality of representations characteristics of portions of the time domain PPG signal to the predetermined quality criteria comprises, for each portion, computing a statistical similarity measure between the portion of the generated time domain PPG signal and previous portions of the generated time domain PPG signal and determining if the computed statistical similarity measure is within the predetermined statistical similarity measure limit.

17. The method of claim 12, further comprising adding representations of portions of the generated time domain PPG signal that meet the predetermined quality criteria to a buffer, while discarding portions of the generated time domain PPG signal that do not meet the predetermined quality criteria, and wherein representations of portions of the generated time domain PPG signal in the buffer are used to generate the digital characterization of the biological characteristic at the point in time.

18. The method of claim 17, further comprising:
identifying a plurality of consecutive portions of the time domain PPG signal that meet additional predetermined quality criteria;
based on identifying a plurality of consecutive portions that meet additional predetermined quality criteria:
purging the buffer; and
filling the buffer with representations of the plurality of consecutive portions; and
wherein generating a digital characterization of the biological characteristic is performed by using the representations of the plurality of consecutive portions in the buffer.

19. The method of claim 18, wherein the additional predetermined quality criteria is a set of criteria with stricter parameters than the predetermined quality criteria.

20. A system for providing a value of a biological characteristic, the system comprising:
one or more hardware processors;
one or more computer readable media coupled to the one or more processors, wherein the one or more computer readable media comprise computer executable instructions that when executed by the one or more hardware processors cause the system to perform the following:
using a sensor, generating a time domain PPG signal;
evaluating portions of the generated time domain PPG signal against predetermined quality criteria; and
using portions of the generated time domain PPG signal that meet the predetermined quality criteria to generate an output value, the output value characterizing a biological characteristic at a point in time.
